(19) United States
(12) Patent Application Publication
(10) Pub. No.: US 2005/0101959 A1
(43) Pub. Date: May 12, 2005

(54) Internal fixator of bones

(76) Inventor: Milorad Mitkovic, Nis (YU)

Correspondence Address: JOHN RUSSELL UREN, 1590 BELLEVUE AVENUE, SUITE 202, WEST VANCOUVER, BC, V7V1A7, CA

(21) Appl. No.: 10/450,056

(22) PCT Filed: Nov. 20, 2001
(86) PCT No.: PCT/YU01/00029

(30) Foreign Application Priority Data
Nov. 22, 2000 (YU) P-730/00

(51) Int. Cl. A61B 17/58

(52) U.S. Cl. 606/69; 606/73

(57) ABSTRACT

Invention is related to the internal bone fixation device by which we solve a problem of fixation and compression of bone fragments, in case of fractures or other operative interventions on bones, in the field of orthopedic surgery. Aim of the invention is to decrease contact between the implant and surface of the bone, providing minimally invasive application of the implant.

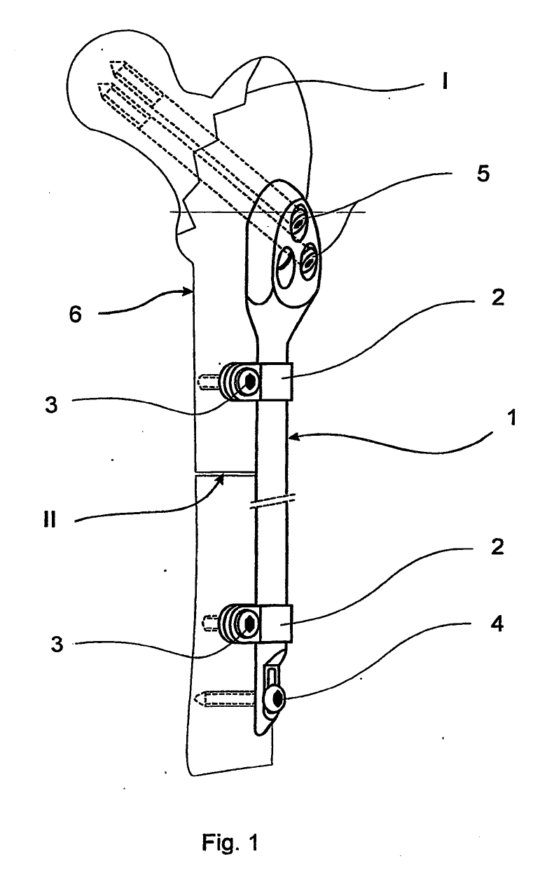

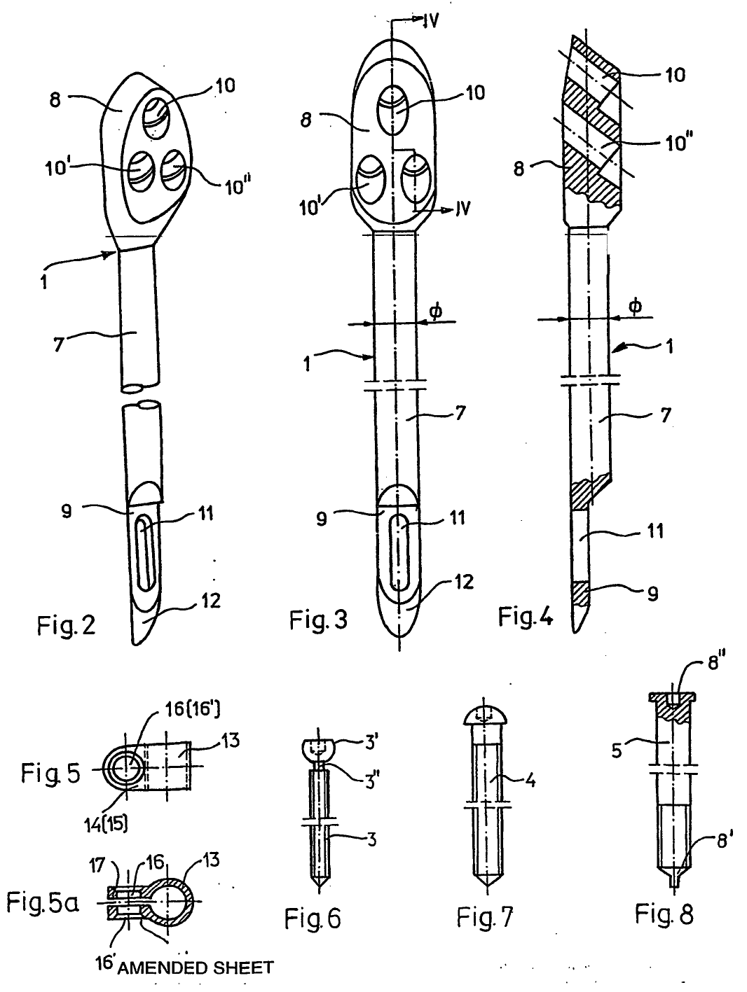

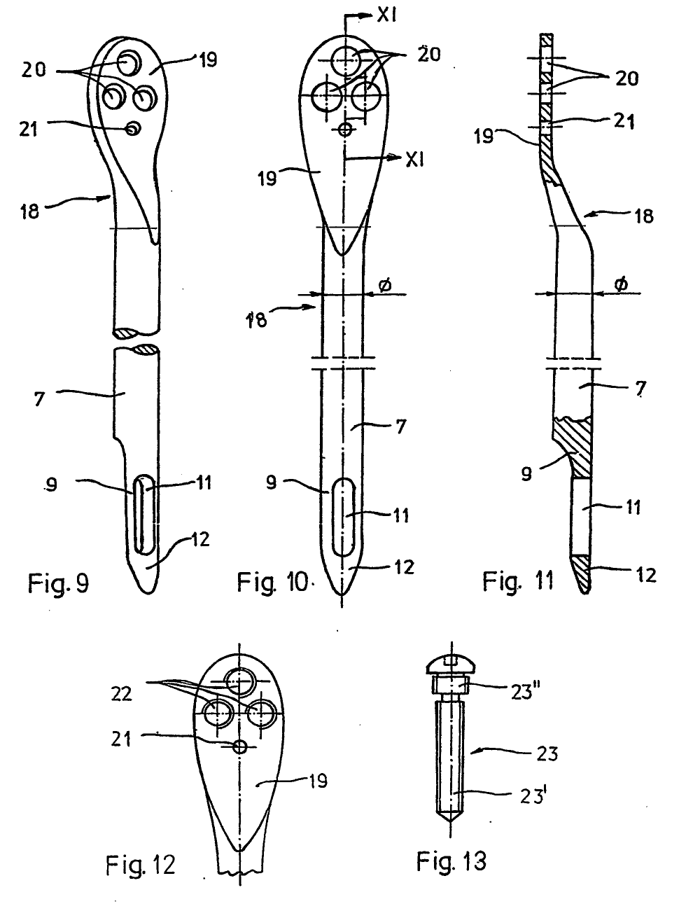

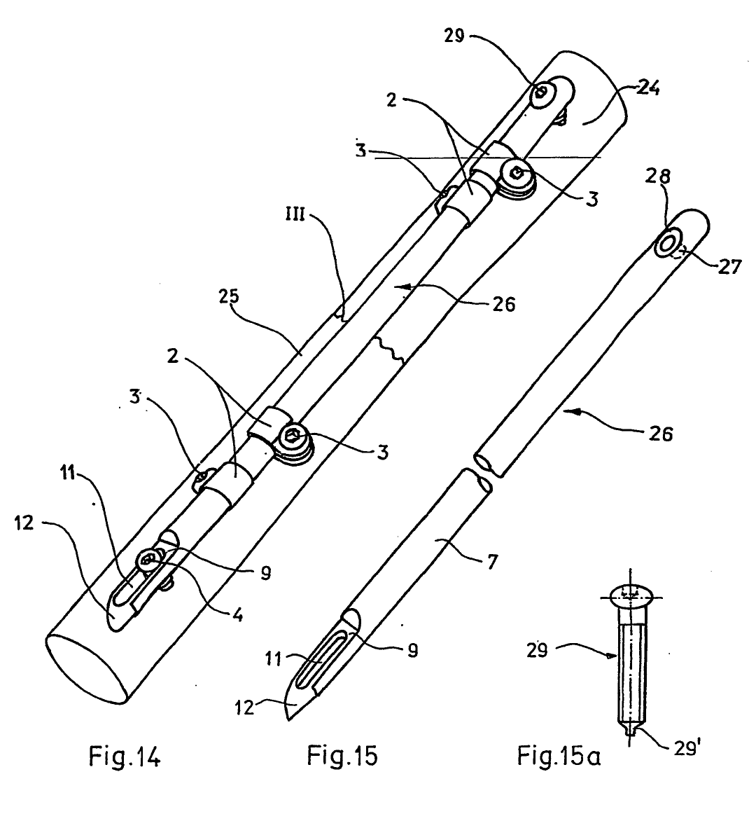

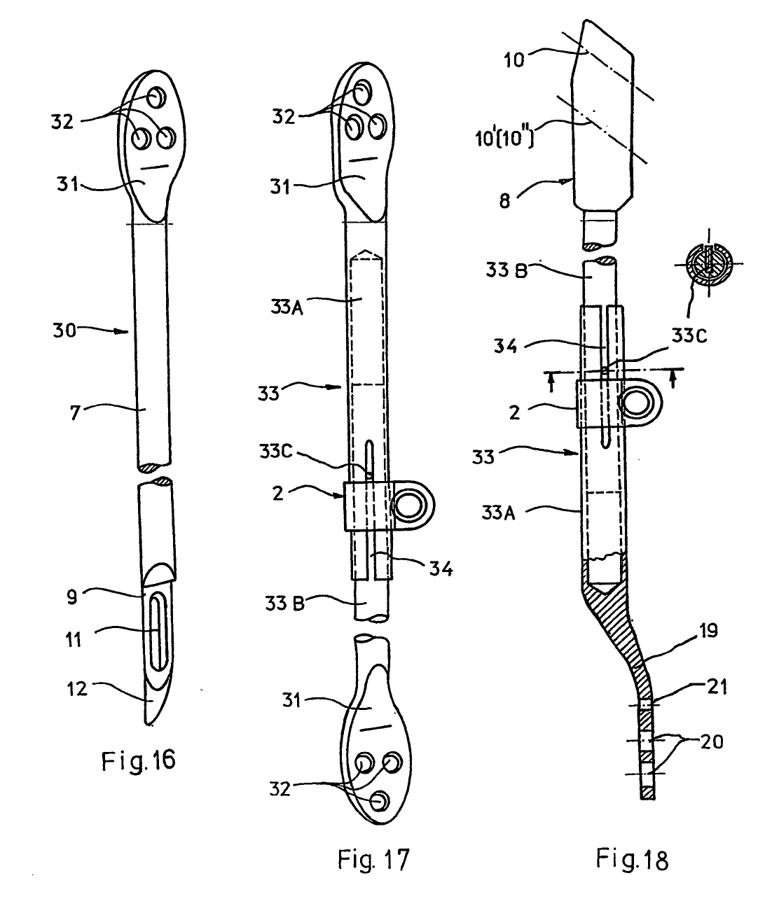

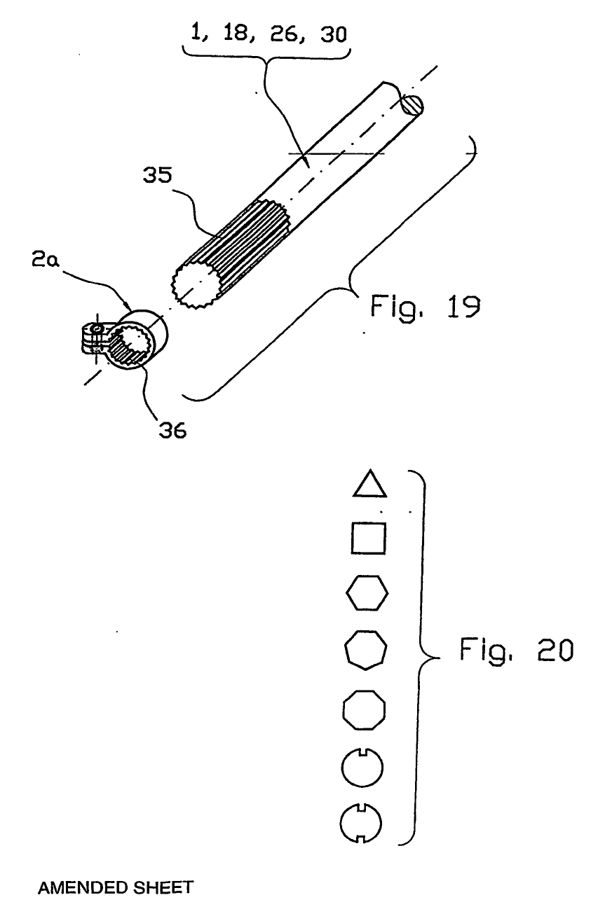

Depending on the type of the bone, several ways of construction of the invention are provided. The basic construction is shown on the example of the femur (6) with fracture (I) close to the hip joint, and fracture (II) on a bone shaft. The invention consists of assembly of connecting rod (1) with clamps (2) and screws (3, 4, 5).

The connecting rod (1) has trochanteric thicker plate (8) with three parallel, slanting, cylindrical holes (10, 10′ 10″) for screws (5) through fracture (I), whereas on the diaphyseal end has flat, thin part (9) with longitudinal slot (11) and rounded tip (12), to accept screw (4). On the cylindrical, rod-like part (7) of the connecting rod (1), two clamps (2) are pulled above fracture (II) and beneath it, with screws (3) that, when screwed into bone fragments, fix the connecting rod (1) with clamp (2) at the same time. The other shown ways of construction of the internal fixation device are condylar, diaphyseal, epiphyso-metaphyseal, telescopic epiphyso-metaphyseal and telescopic trochanteric-condylar fixator.

### TECHNICAL FIELD THAT INVENTION IS RELATED TO

Technical field that the invention is related to is, generally saying, a medical science field, i.e. orthopedic surgery, wherein, a subject of the invention is related to the internal bone fixation device.

According to the International Patents Classification (MKR⁷), a subject of the invention is designated by the basic classification symbol A 61 B 17/68 used for designation of internal bone fixation (osteosyntesis) devices that are used in orthopedic surgical methods for treatment of bones and joints fractures.

### TECHNICAL PROBLEM

Technical problem to be solved by the subject of this invention is the following: How to design the internal bone fixation device that, when used, can solve a problem of bone fixation and compression in case of fracture or other surgical intervention on bones in orthopedic surgery, thus preventing movement of bone fragments (except due to the possible compression) after their fixation, and decreasing contact between an implant and the bone solving the problem of preservation of periosteal, intramedular and intraosseal bones blood circulation.

### STATUS IN TECHNIQUE

Attempts of internal bone fixation device construction in Switzerland are known, but that device has neither possibility of compression, nor axial dynamic compression.

In American patent No. U.S. Pat. No. 4,887,595 are shown a device that is implanted surgically for maintaining a relative position and firmness of spinal bones. It consists of longitudinal and widened plate-shaped body that extends integrally into a narrowed rod-shaped part. Two cuts are made coaxially through the plate-shaped body to accept a component that will fix the plate-shaped part to the parts of spinal bones. On the upper surface of these longitudinal cuts there are holes, for example, six holes at each cut, used to accept screws on the neighboring parts of spinal bones that need to be fixated. The rod-shaped part can be bended according to the configuration of spinal cord parts, wherein, the rod-shaped part is fixed to the spinal bones, through cylindrical clamps, by means of screws.

Universal eccentric mechanism for bone fixation in orthopedic surgery is shown in American patent No. U.S. Pat. No. 5,575,791. It presents a new way of tight connection of longitudinal, spherical rod with coupling that has inserted eccentric ring with cuts. The eccentric ring can be cylindrical or conical, and they are inserted in the adequate spherical, ring-like or conical space in the center of a coupling through which is dragged a screw for placing into the bone fragment.

American patent No. U.S. Pat. No. 3,842,825 describe a hip fixation device, or in other words, device for reconstruction of fragment of the upper hip end fragments. The device consists of a strip-like rod, symmetrically widened at the trochanteric end of the femur, taking shape of a thin plate. Four elliptical holes are made in the plate, with guides placed outwards for passing screws that should be inserted into the mentioned bone fragments. Screw heads are covered by halfsphere-like cover fixed to the thin plate. Through a strip-like rod are alternately made spherical holes used for connecting the device with bones.

There are some more foreign patents from the subject field, such as U.S. Pat. No. 5,484,439; U.S. Pat. No. 2,486,303; U.S. Pat. No. 4,854,311; PCT/EP/95/03494; PCT/EP/99/09065 that are related to the devices and instruments for bone fixation, and that could be relevant in relation to the internal bone fixation device according to the invention described herein.

However, all the above-mentioned solutions of internal and external bone fixation devices have two basic disadvantages. The first is, that they are mostly plate-shaped, and that, after being applied and fixed, decrease remarkably a periostealy blood circulation. The other disadvantage concerns the impossibility of sliding movement of the connecting rod along fragments, by which, so called, axial dynamic compression becomes spontaneously possible. Both disadvantages are eliminated by the newly suggested solution according to the subject of patent registration, and they will be described in details in the following text, and shown on the drawings attached herewith.

### DESCRIPTION OF THE INVENTION'S ESSENCE

Internal bone fixation device, according to this invention, represents an innovation in solving problem of bone fixation and compression at fracture or other surgical interventions on bones in orthopedic surgery field. The essence of the invention is to decrease a contact between implant and a bone surface, from one side, thus solving a problem of blood circulation through the bone, and to prevent bigger movement of bone fragments (except due to the compression) after fixation, from the other side but also to be suitable for minimally invasive application.

The invention consists of a connecting rod, at least two clamps and three types of screws that are placed through connecting rod and clamps into the bone fragments. All elements of the internal bone fixation device are manufactured from highly alloyed steel or titanium. Depending on the types of bones, several ways of the internal bone fixation device are provided according to this invention.

According to the basic way of construction used on the example of femur, with the fracture near to the hip joint and with eventual the second fracture on the bone shaft, internal trochanteric bone fixation device is provided. It consists of connecting rod integrally made in several different lengths, for example, from 150 mm-400 mm. The connecting rod has a cylindrical rod-like part extended at its trochanteric end to a thick plate in which three cylindrical holes are pierced diagonally upwards, parallel to ach other. Through those holes are inserted long, plate-like headed screws with arrow-shaped tip to ease screwing in the neck and head of femur. On the opposite diaphyseal (shaft) end of the connecting rod is made a thin part, perpendicular from the top, with longitudinal slot for inserting screw with semi-lens head in it, that is placed beneath the second fracture on the bone shaft. Before that, two ring-like clamps were pulled over the cylindrical rod-like part of the connecting rod, and they are fixed to the bone surface in proximally of the eventual second fracture and distally of it, by ball-like headed screws.

The other ways of construction of the internal bone fixation device are also provided according to this invention, and they are: condylar, diaphyseal, epiphyseo-metaphyseal, epiphyseal telescoping and trochanteric-condylar telescopic internal fixator.

### BRIEF DESCRIPTION OF THE DRAWINGS

In order to understand the invention easily, and to show how it can be realized in practice, the author refers to the project drawings attached herewith and related to the internal bone fixation device, wherein:

Drawing 1 shows a complex look of trochanteric internal bone fixation device applied on femur fractures

Drawing 2 shows look of connecting rod of trochanteric internal bone fixation device of the drawing 1, observed in axonometric view

Drawing 3 shows look of the connecting rod of fixation device of the drawing 2, observed from its front side in vertical projection

Drawing 4 shows look of fixation device connecting rod in vertical, partial cross section, done at the line IV-IV of the drawing 3

Drawing 5 shows lateral look of clamp for fixing connecting rod of the fixation device to the bone fragments, observed in vertical projection

Drawing 5a shows look of the clamp of the drawing 5, observed in horizontal projection

Drawing 6 shows look of a spherical-headed screw, used through the clamp and bone fragments

Drawing 7 shows look of a screw with half lens-like head for connecting the bone fragments through the bone shaft end of the internal fixation device connecting rod

Drawing 8 shows look of a screw with cylindrical, plate-like head and arrow-shaped tip for connecting fragments close to the hip and knee joint with widened trochanteric end of the internal fixation device body

Drawing 9 shows axonometric look of connecting rod of condylar internal fixation device

Drawing 10 shows frontal look of connecting rod of the drawing 9, observed in vertical projection

Drawing 11 shows look of connecting rod in vertical section, done at the line XI-XI of the drawing 10

Drawing 12 shows look of the widened condylar end of the internal fixation device according to one more way of construction, comparing to the one shown on the drawing 10

Drawing 13 shows look of the lens-like headed screw and two-part thread for connection of bone fragments with condylar end of the drawing 12

Drawing 14 shows an axonometric complex look of the internal fixation device applied on bone fragments longitudinally

Drawing 15 shows an axonometric look of connecting rod of the internal fixation device of the drawing 14

Drawing 15a shows a ball-like headed screw applied as a part of the internal fixation device of the drawing 14

Drawing 16 shows axonometric look of connecting rod of uniepiphyseal or unimetaphyseal internal fixation device

Drawing 17 shows axonometric look of biepiphyseal or bimetaphyseal telescopic internal fixation device

Drawing 18 shows lateral look of the connecting rod of trochanteric-condylar telescopic internal fixation device

Drawing 19 shows axonometric look of assembly of diaphyseal end with clamp, according to the second way of construction

Drawing 19a shows axonometric look of assembly of diaphyseal end with telescopic insert which comprises plate

Drawing 20 shows seven more diaphyseal ends of rods and clamps of different geometric shapes, according to the drawing 19

### DETAILED DESCRIPTION OF THE INVENTION

By observing drawing 1 attached herewith, we can note that the internal bone fixation device, according to this invention, consists of connecting rod 1, at least two clamps 2 and screws 3, 4 and 5. It is applied as an example on the femur 6, with fracture I close to the hip joint, and fracture II on the bone shaft.

The connecting rod 1 of the internal fixation device that is separately shown on the drawings 2, 3 and 4 of the project attached herewith, represents an integral assembly. It is manufactured in several lengths of the highly alloyed steel or titanium. It consists of a cylindrical, rod-like part 7 that has on its trochanteric end a widened thick plate 8, and on the other bone shaft end, from the upper side, it has a thin part 9 which is, from one side, flattened bar 7. In the plate 8, three identical cylindrical holes 10, 10′ and 10″ are pierced diagonally upwards, in a triangular disposition, wherein, their central axis are parallel and in relation to the longitudinal axis of the cylindrical, rod-like part 7, form an angle of 120°-140°. Cylindrical openings 10, 10′ and 10″ are used for dragging screws 5 that should be screwed in a femoral neck and head, at location of fracture I, close to the hip joint, as per drawing 1. Look of the screws 5 is shown on the drawing 8, and their characteristic are that they have cylindrical plate-like heads, relatively short thread comparing to their lengths, and arrow shaped tip 8′. Through the center of the screws 5, from their upper side, is pierced a hexagonal opening 8″ used for placing a corresponding screwdriver in it, to screw in the screws 5.

In the flat thin part (9) at the shaft end, a longitudinal slot 11 is made for placing screw 4 in it, wherein, the length of the slot 11 exceeds diameter of the screw 4, several times. The screw 4 has lens-like head in which a hexagonal opening is pierced for screwing in. Tip 12 of the thin part 9, is narrowed from both sides, and rounded to ease placing of connecting rod 1 through a skin incision and muscle tissue.

Clamp 2 is shown separately on the drawings 5 and 5a of the drawing attached herewith, and it has a role to connect the connecting rod 1 with femur 6, in indirect way. The clamp 2 has a cylindrical ring 13 cut along its height, wherein, plate-shaped guides 14 and 15 are integrally pointed out at the ends of the cut. Through the center of the guides 14 and 15, identical cylindrical holes 16 and 16′ are coaxially pierced, and their diameters exceed a diameter of the screw 3. Cylindrical holes 16 and 16′, at the outer ends of holes, have identical spherical or conical widened parts 17 and 17′, assigned for leaning on of the spherical head 3′ of the screw 3. In order to ease approach to the screw 3 for its screwing adjustment, a neck 3″ is made on it, with diameter less than outer diameter of its threads. Length of the neck 3″ equals the sum of thicknesses of plate-shaped guides 14 and 15, while the inner diameter of the cylindrical clamp 13 is slightly exceeding the diameter of the cylindrical, rod-shaped part 7 of the connecting rod 1. It is shown up on the drawing 1 that clamps 2 are pulled over the connecting part 7, above the lower fracture II of the femur 6, and beneath it. Screw 3 has also, on the upper side of its ball-like head 3′, pierced hexagonal opening for screwdriver in, similar to those of the screws 4 and 5, whereby screw 5 can be canulated. Between the clamp 2 and the head 3′ of the screw 3 can be inserted biodegradable ring. After degradation of this ring the clamp 2 becomes loose allowing so sliding of the connecting rod 1, 18, 26, 30 that will have as a consequence compression on the fracture site produced by the muscle force, which leads to better bone healing.

The other way of construction of the internal fixation device, according to this invention, is related to the condylar internal fixation device, with a new construction of the connecting rod showed on the drawings 9, 10 and 11 of the project attached herewith. This way of the internal fixation device construction is especially suitable for fixation of the upper and lower femur, and for the other bones as well. Modification is related to the condylar end that is made as elliptically elongated, and relatively thin plate 19 extended outwards, and parallel, comparing to the cylindrical rod-like part 7 (see the drawing 11). Through a plate 19, three or more identical cylindrical holes 20 are pierced in triangular disposition, with one more cylindrical hole 21 pierced beneath them, with a smaller diameter. Through cylindrical holes 20, screws 5 are introduced to be screwed into the bone. The cylindrical holes 20 might be slightly slanted outwards (from 90°-95°) in relation to the longitudinal axis of the cylindrical, rod-like part 7.

Lower, shaft end of the second way of construction is practically the same as the one of the trochanteric internal fixation device described before, so, it is not necessary to describe its construction repeatedly.

Instead of three or more identical cylindrical holes 20 on the plate 19, only one or two cylindrical holes 20 can be made in it, depending on the location of fracture on the femur or on some other bone.

On the drawing 12 of the project attached herewith, cylindrical threading holes 22 are shown, instead of cylindrical holes 20, and specially made screws 23, must be screwed in them. Characteristic of those screws 23 is, that they have two separated threading parts, i.e. longer threading part 23′ with smaller diameter for screwing in the bone, and short threading part 23″, for screwing into threading holes 22. Shorter threading part 23″ is made directly beneath a half lens-like head with hexagonal opening, for screwing in, and its thread length is slightly exceeding thickness of the plate 19.

Also, instead of cylindrical threading holes 20, conical threading holes can be made through plate 19 (not shown on the drawing). In that case, shorter threading part 23″ of the screw 23 must be made with a conical thread on the head of the screw.

The third way of internal bone fixation device construction, according to this invention, is related to the diaphyseal internal bone fixation device that is used for the shaft of the bone, while it is not used on the ends of bones. That type of fixation device is showed on the drawing 14 attached herewith, as one of the examples of its application at fracture III, i.e. for connection of fragments 24 and 25 with connecting rod 26. On the drawing 15, connecting rod 26 is showed separately, and according to this way of construction, it is a flat rod with spherical cross section, manufactured in several different lengths, depending on the need. In some cases, connecting rod 26 might be slightly bended, depending on the shape of the bone to be applied on. One end of the connecting rod 26 is rounded in a shape of a hemisphere, and close to it is pierced cylindrical hole 27, providing that longitudinal axis of the connecting rod 26 and hole 27 form an angle of 90°. The cylindrical hole 27 has on its upper end a conical or spherical wider shape 28 for leaning on the ball-like head of the screw 29 shown on the drawing 15a attached herewith. Its tip 29′ is arrow-shaped to ease its screwing in. The other end of the connecting rod 26 is made in the same way as on the connecting rods 1 and 18 on the shaft ends of previous ways of construction, i.e. with slot 11 on the flat, thin part 9, and rounded tip 12. Through the slot 11, screw 4 is introduced and screwed into the bone.

From the drawing 14 it can be observed that four clamps 2 are pulled over the connecting rod 26 by sliding, and are arranged in pairs on bone fragments 24 and 25, wherein, clamps 2 are also fixed to the connecting rod 26 by means of screws 3 inserted through the bone fragments 24 and 25. The most optimal arrangement of clamps 2, either individually or in pairs, on bone fragments 24 and 25, can be achieved on the basis of experience, taking in consideration location of the fracture III.

When the diaphysal (shaft) internal fixation device is fixed to the fragments 24 and 25 on the above-described way, and as per drawing 14, it will tightly catch the fracture position. If clamps 2 on the fragment 24 are slightly loosen, they will lose their tight connection with connecting rod 26 that can slide through those clamps 2. And if at location of the fracture III exists a gap between fragments 24 and 25, a compression force will cause sliding of the connecting rod 26 along slot 11, and in direction dictated by screw 4 screwed in the fragment 25. Accordingly, bone fragment (24) slides towards the fragment 25 causing compression on the location of the fracture III. Screws 4 and 29, at the ends of the connecting rod 26 provide rotating stability of the bone fragments 24 and 25 they are screwed in.

On the drawing 16 is showed connecting rod 30 of epiphyseal or metaphyseal (end part of each long bone) internal bone fixation device that has similarities to the condylar connecting rod 18 of the drawing 9. Cylindrical, rod-like part 7 is extended continually into elliptical, elongated, relatively thin plate 31, with three or more identical spherical holes 32, in triangular disposition. The other end of the connecting rod 30 is made in the same way, except that it is rounded for 180°, similar to the connecting rods 1, 18 and 26 on their shaft ends. Characteristic of the connecting rod 30 is, that a thin plate 31 is slightly positioned outwards, and parallel to the rod-like part 7, and that instead of three spherical holes 32, on the thin plate 31, there can be two, or only one hole. For the connection of the connecting rod 30 with the bone fragments are used already mentioned screws 3 or 4, in addition to, at least, one clamp 2 with screw 3. Connecting rod 30 might be slightly inclined by adequate bending, in order to be used in special cases. The plate 31 is not in the same level as rod 30 but it has prominence of its bellow side for the thickness of the clamp.

Internal fixation device with telescopic insert 40 showed on the drawing 19a has a connecting rod 1, 18, 26, 30 which has on the diaphyseal end different shape with cross sections such as triangular, square, pentagonal, hexagonal octagonal, star-like, cylindrical with or without one or two longitudinal groves or prominences, as well as the other regular or irregular shapes on cross section. Al these parts are as mail. The inner surface 38 of the pipe-like ending 39 of the insert 40, as female part, must be provided with adequate hole for telescoping connection, whereby, the opposite end of the insert is extended continuously into elliptic, “T” or other shape thin plate 41, which can be bended according to the bone shape and with at list one hole 42 and maximum several holes with or without threads inside the holes. Thread inside the hole can be conical to accept adequate screw with conical thread on its head.

Telescoping internal bone fixation device showed on the drawing 17 has a connecting rod 33 made of two parts. The outer, pipe-like part 33A of the connecting rod 33 has one or two longitudinal cuts 34 (opposite to each other), with integrally extended thin plate 31, with cylindrical holes 32 on its opposite, upper side. The inner cylindrical, rod-like part 33B of the connecting rod 33 has at its end the identical thin plate 31 with cylindrical holes 32. The rod-like part 33B is inserted in pipe-like part 33A, and their tight connection is achieved by means of clamp 2 that is tightened by screws 3 at the location of cut 34. The plate-like prominence 31 has the same prominence as described above. To prevent rotation of rod-like part 33B inside the pipe-like part 33A, one anti rotation pin 33C is fixed to the rod-like part 33B.

Telescoping trochanteric-condylar internal fixation device shown on the drawing 18, has connecting rod 33, made up of two parts as well. The outer pipe-like part 33A of the connecting rod 33 with longitudinal cut 34 has on its opposite end continually extended thin part 9. The inner cylindrical, rod-like part 33B of the connecting rod 33 has on its trochanteric end elongated, thick plate 8, with three diagonally made cylindrical holes 10, 10′ and 10″, parallel to each other, for screws 5. The tight connection between parts 33A and 33B of the connecting rod 33 is achieved by means of clamp 2 and screw 3.

And finally, connecting rods 1, 18, 26 and 30 could have on their shaft (diaphyseal) end, rod-like ending of various cross sections, instead of thin part 9, with longitudinal groove 11 and rounded tip 12. On the drawing 19 is shown ending 35, symmetrically toothed along its parameter, similar to the cog-wheel, wherein, the inner surface of the cylindrical clamp of the clamp 2 is made with toothing 36, that corresponds to the toothing at the ending 35. Connection is realized by pulling such toothed coupling 2a on the toothed end 35 of the connecting rod.

Instead of toothed ending 35 could be made endings of various cross sections, shown on the drawing 20, such as triangular, square, pentagonal, hexagonal, octagonal, spherical with one rectangular groove, spherical with two rectangular grooves, opposite to each other, as well as the other regular or irregular shapes of cross sections. It is understood that the clamp 2 must be adapted according to those cross sections, thus preventing rounding of connecting rods 1, 18, 26 and 30 in clamps 2a, and enabling their axial, sliding movement in case of no so strong tightening of the screw 3. In cases when the screw 3 is firmly tightened through bone fragments and coupling 2a, there will bi neither sliding, nor twisting of the connecting rod.

### THE WAY OF INDUSTRIAL OR OTHER APLICATION OF THE INVENTION

The way of the invention application is obviously deducible from the previous description and project drawings attached herewith, so, there is no need to describe it separately.

1. The internal bone fixation device, assigned in orthopedic surgery for bone fragments fixation, for example femur (6) with fracture (I) close to the hip joint, and fracture (II) on the bone shaft, pointed by that, that contains connecting rod (1), clamp (2) and screws (3, 4, 5), that connecting rod (1) is made as an integral assembly, on which rounded cylindrical, rod-like part (7) at the trochanteric end is continually extended into a thick plate (8), whereas, on the other shaft end it has flattened part (9), and that through the thick plate (8), three identical, cylindrical holes, parallel to each other (10, 10′, 10″) are pierced diagonally upwards, under the angle of approximately 130°, and in triangular disposition, wherein, through the cylindrical holes (10, 10′, 10″) are dragged rounded, plate-shaped headed screws (5) with hexagonal opening (8′) pierced in, and arrow-shaped tip (8″), that screws (5) are inserted through neck and head of the bone, in case of fracture (I) of femur (6), and that through the flat, thin part (9) is made longitudinal slot (11) for introducing half lens-like headed screw (4), whereas a tip (12) of the thin part (9) is narrowed and rounded from both sides, whereby screw (5) can be canulated.

2. The internal bone fixation device, according to the claim 1, pointed by that, that the clamp (2) has cylindrical ring (13) cut along its height, with plate-like guides (14, 15), integrally positioned outwards, and parallel to each other, and that in their center are pierced coaxial, cylindrical holes (16, 16′) with conical widening (17, 17′) on their outer sides, for leaning on of the ball-like head (3′) of the screw (3), wherein, between the thread and ball-like head (3′), on the screw (3) is constructed narrowed neck (3″), that clamps (2) are, by their cylindrical rings, pulled on the cylindrical, rod-like part (7) of the connecting rod (1), and fixed by means of screws (3) placed above fracture (II) and beneath it, through the shaft of the femur (6), and that, in the assembly between the shaft of the femur (6) and rounded cylindrical, rod-like part (7) of the connecting rod, is achieved a minimal distance.

3. The internal bone fixation device, according to the claim 1, and modification I, especially applicable for fixation of the upper and lower ends of the femur (6), and other bones as well, pointed by that, that the condylar end of the connecting rod (18) is made as the elliptical, longitudinal, thin plate (19), partially positioned outwards in relation to the cylindrical, rod-like part (7), while its shaft end has a flat, thin part (9) with longitudinal slot (11) and narrowed and arch-like rounded tip (12), that through the thin plate (19) are pierced in triangular disposition three identical holes (20), and a smaller hole pierced proximally to them, wherein, screws (5) are inserted in cylindrical holes (20), and that, instead of three cylindrical holes (22) on the thin plate (19), three cylindrical or conical threading holes can be made, and through them are fixed half lens-like headed screws (23), with longer threading part (23′) with smaller diameter, and shorter threading part (23″) with bigger diameter, or screw with conical thread on its thread.

4. Internal bone fixation device according to the claim 3, pointed by that, that through elliptical, elongated, thin plate (19), only one or two or more identical cylindrical holes (20) are pierced for screws (5), i.e. one or two cylindrical or conical threading holes (22) for screws (23) or for screw with conical thread on its head.

5. The internal bone fixation device, according to the claim 1, and modification II, especially applicable on the central parts of bones, i.e. on the long bone shaft, such as fracture (III), for connecting the bone fragments (24, 25), pointed by that, that shaft internal connecting rod (26) is made up of strait, or slightly bended round rod that has, near to its rounded end pierced vertical, cylindrical hole (27) with conical widening (28), coaxially made on its upper side, for receiving a screw (29) with spherical head and arrow-shaped tip (29′), that the other end of connecting rod (26) is made up as flat, thin part (9), with rounded tip (12), and longitudinal slot (11), to accept screw (4), and that on the connecting rod (26) are pulled clamps (2), arranged in pairs on bone fragments 24 and 25, above fracture (III) and beneath it, wherein, clamps (2) are fixed by means of screws (3) screwed in bone fragments (24, 25), whereby between the clamp (2) and the head (3′) of the screw (3) can be inserted biodegradable ring.

6. Internal bone fixation device, according to the claim 1, and modification III, specially made as epiphyso-metaphyseal bone fixation device, pointed by that, that it has connecting rod (30) with cylindrical, rod-like part (7) extended continually into elliptical, elongated, thin plate (31), with, at least, one and maximum several cylindrical holes (32), in triangular or other disposition, wherein, the thin plate (31) is parallel and positioned slightly outwards for the distance of the thickness of the wall of the clamp (13), in relation to the cylindrical rod-like part (7), that on diaphyseal end of the connecting rod (30) is made flat, thin part (9), with longitudinal slot (11) and rounded tip (12), and that the cylindrical, rod-like part (7) of the connecting rod (30) can be slightly bended.

7. Internal bone fixation device, according to the claims 1, 3 and 6, and modification IV, especially used as a telescopic internal fixation device, pointed by that, that the end of the pipe-like part (33A) of the connecting rod (33) is extending continually into a flat, thin part (31) or into thin plate (19), whereas the end of the internal cylindrical, rod-like part (33B) is continually extended into a widened and thick plate (8) or the thin plate (31), that at the middle of the cut (34), and on the outer pipe-like part (33A) is fixed clamp (2) with screw (3), and in order to prevent rotation of rod-like part (33B) inside the pipe-like part (33A), one anti rotation pin (33C) is fixed to the rod-like part (33B)

8. Internal bone fixation device, according to the claims 1, 2, 3, 4, 5 and 6, and modification V, especially used as a internal fixation device with telescopic insert, pointed by that, that diaphyseal end of connecting rods (1, 18, 26, 30) can be of different shapes with cross sections such as triangular, square, pentagonal, hexagonal octagonal, star-like, cylindrical with or without one or two longitudinal groves or prominences, as well as the other regular or irregular shapes on cross section, and that inner surface (38) of the pipe-like ending (39) of the insert (40) must be provided with adequate hole for telescoping connection, whereby, the opposite end of the insert is extended continuously into elliptic, “T” or other shape thin plate (41), which can be bended according to the bone shape and with at list one hole (42) and maximum several holes with or without threads inside the holes.

9. Internal bone fixation device, according to the claims from 1 to 7, and modification VI, pointed by that, that diaphyseal end of connecting rods (1, 18, 26, 30) is made as rod-like, toothed ending (35), whereas, the inner surface of the cylindrical ring (13) of the clamp (2a) is made with the adequate inner toothing (36), wherein, clamp (2a) is pulled on the toothed ending (35) of the connecting rods (1, 18, 26, 30).

10. Internal bone fixation device according to the claims 8 and 9, pointed by that, that diaphyseal ends of connecting rods (1, 18, 26, 30) can be of different shapes, with cross sections such as triangular, square, pentagonal, hexagonal, octagonal, cylindrical with one longitudinal, groove or prominence, cylindrical with two longitudinal, grooves or prominences opposite to each other, as well as the other regular and irregular shapes on cross sections, and that ring (13) of clamp (2a) must be provided with adequate inner hole for connecting.
